(19) United States
(12) Patent Application Publication
(10) Pub. No.: US 2026/0279554 A1
(43) Pub. Date: Sep. 17, 2026

(54) HEALTH CONDITION CHANGE VALIDATION

(71) Applicants: Brajendra S. Thakur, Pittsburgh, PA (US); Nancy Hunsberger, Mount Laurel, NJ (US); Nicole Blohm, Mount Pleasant, SC (US); Stephen Cousins, Nashville, TN (US)

(72) Inventors: Brajendra S. Thakur, Pittsburgh, PA (US); Nancy Hunsberger, Mount Laurel, NJ (US); Nicole Blohm, Mount Pleasant, SC (US); Stephen Cousins, Nashville, TN (US)

(73) Assignee: Ricoh Company, Ltd., Tokyo (JP)

(21) Appl. No.: 19/079,290

(22) Filed: Mar. 13, 2025

(51) Int. Cl. G16H 40/20; G16H 10/60; G16H 40/63

(52) U.S. Cl. CPC G16H 40/20; G16H 10/60; G16H 40/63

(57) ABSTRACT

Disclosed herein are techniques related to health condition change validation. In some embodiments, the techniques may involve receiving measurement data from one or more first networked devices for a patient being monitored. Furthermore, the techniques may involve receiving mental condition data of the patient. The techniques may also involve detecting, based on analyzing the measurement data from the one or more first networked devices, a change in a physical/physiological condition of the patient. Additionally, the techniques may involve determining, based on the mental condition data and the change in the physical/physiological condition of the patient, that the change in the physical/physiological condition of the patient warrants communicating, to a first recipient, a first notification of the change in the physical/physiological condition of the patient. The techniques may further involve communicating, toward the first recipient, the first notification of the change in the physical/physiological condition of the patient.

### FIELD OF THE INVENTION

This disclosure relates generally to the medical arts, and more specifically, to health condition change validation.

### BACKGROUND

The approaches described in this section are approaches that could be pursued, but not necessarily approaches that have been previously conceived or pursued. Therefore, unless otherwise indicated, the approaches described in this section may not be prior art to the claims in this application and are not admitted to be prior art by inclusion in this section.

The Internet revolution opened a world of possibilities by enabling communication between devices that had previously operated in isolation. Of particular note is the emergence of monitoring systems composed of different devices (e.g., cameras, microphones, and/or sensors) that can share information such that the resulting mosaic of shared information provides a deeper understanding of the monitored subject. Furthermore, when response systems (e.g., 911 emergency response system and/or nurse station response system) can receive real-time notifications of circumstances detected by monitoring systems, the response systems become empowered to respond effectively.

However, delivering notifications indiscriminately can reduce the effectiveness of a response system. For example, sending all notifications to both a 911 emergency response system and a nurse station response system can have the effect of a distributed denial of service (DDoS) attack in which both systems are inundated with unnecessary notifications. On the other hand, failing to notify a response system can also have adverse consequences. For example, failing to notify a nurse station response system that a newborn baby has not been eating can result in jaundice or even death.

Thus, there is a need for a notification system that does not suffer from the aforementioned shortcomings.

### BRIEF SUMMARY

Disclosed herein are techniques related to health condition change validation. The techniques may be practiced with a computer-implemented method; a computer-readable medium storing instructions which, when executed by one or more processors, cause performance of the computer-implemented method; and/or a system comprising a memory storing instructions which, when executed by one or more processors, cause performance of the computer-implemented method.

In some embodiments, the techniques may involve receiving measurement data from one or more first networked devices for a patient being monitored. Furthermore, the techniques may involve receiving mental condition data of the patient. The techniques may also involve detecting, based on analyzing the measurement data from the one or more first networked devices, a change in a physical/physiological condition of the patient. Additionally, the techniques may involve determining, based on the mental condition data and the change in the physical/physiological condition of the patient, that the change in the physical/physiological condition of the patient warrants communicating, to a first recipient, a first notification of the change in the physical/physiological condition of the patient. The techniques may further involve communicating, toward the first recipient, the first notification of the change in the physical/physiological condition of the patient.

### BRIEF DESCRIPTION OF THE DRAWINGS

In the figures of the accompanying drawings like reference numerals refer to similar elements.

FIG. 1 is a block diagram that depicts an example architecture for intelligent notification delivery, according to embodiments.

FIG. 2 is a block diagram that depicts an example context data source, according to embodiments.

FIG. 3 is a block diagram that depicts an example notification system, according to embodiments.

FIG. 4 is a flow diagram that depicts an example approach for performing intelligent notification delivery, according to embodiments.

FIG. 5 is a flow diagram that depicts an example approach for determining whether or not a notification is warranted, according to embodiments.

FIG. 6 is a block diagram of an example software system that may be employed for controlling the operation of a computing device, according to embodiments.

FIG. 7 is a block diagram of an example computer system on which embodiments may be implemented.

### DETAILED DESCRIPTION

In the following description, for the purposes of explanation, numerous specific details are set forth in order to provide a thorough understanding. It will be apparent, however, to one skilled in the art that some embodiments may be practiced without these specific details. Well-known structures and devices are shown in block diagram form in order to avoid unnecessarily obscuring the disclosure. Various aspects of the disclosure are organized hereinafter into the following sections:
I. OVERVIEW
   II. INTELLIGENT NOTIFICATION DELIVERY ARCHITECTURE
      A. CONTEXT DATA SOURCE
      B. NOTIFICATION SYSTEM
      C. RECIPIENT DEVICE(S)
   III. APPROACHES FOR INTELLIGENT NOTIFICATION DELIVERY
   IV. IMPLEMENTATION MECHANISMS

### I. Overview

Disclosed herein are techniques related to health condition change validation. Among other things, the techniques leverage voice/text input analysis to validate a detected change in physical/physiological condition of a patient. In some embodiments, the techniques also leverage natural language processing to detect a change in mental condition of the patient, and the detected change in mental condition may be used to validate the change in physical/physiological condition.

To illustrate, suppose that Sarah is a patient undergoing at-home monitoring. When Sarah's glucose monitor detects a glucose level of 100 mg/dL in the early afternoon, Sarah's smartphone asks her if she skipped lunch. Sarah responds with voice input confirming that she skipped lunch. A paralinguistic analysis of the voice input suggests that Sarah is suffering from depression. Sarah's husband is notified that Sarah's glucose level is low and that she may be depressed. Sarah is also asked for permission to notify a psychologist about her depression.

Thus, the techniques reduce the amount of unnecessary notifications by increasing the accuracy of detecting changes in health conditions. This can have the technical effect of reducing bandwidth consumption and otherwise conserving computing resources.

### II. Intelligent Notification Delivery Architecture

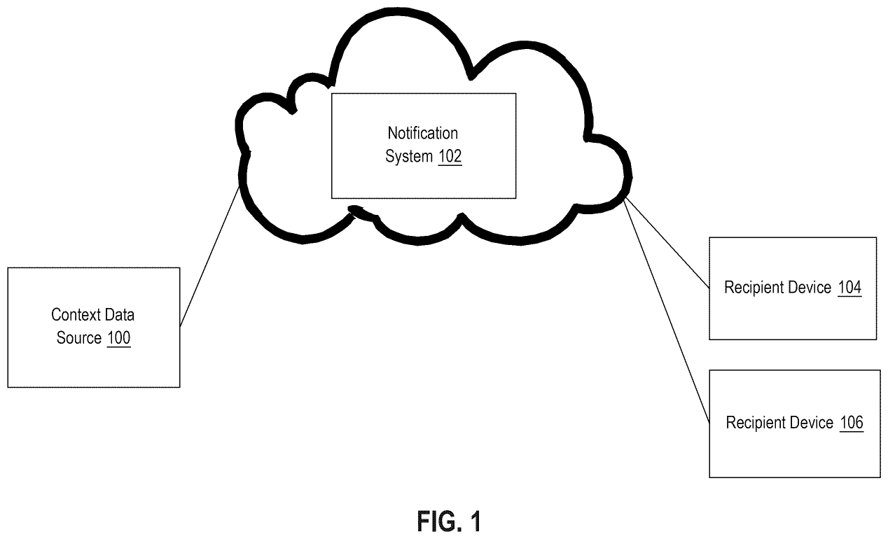

FIG. 1 depicts an example architecture for intelligent notification delivery. The example architecture includes context data source 100, notification system 102, and recipient devices 104-106. In FIG. 1, notification system 102 is depicted as a cloud-based system that is communicatively coupled to context data source 100 and recipient devices 104-106.

### A. Context Data Source

Context data source 100 may be a system of one or more computing devices configured to provide context data to notification system 102. Context data refers to information that can be used to determine the condition (e.g., health condition and/or physical condition) of a subject (e.g., a patient, a vehicle, or some other object of interest). As used herein, a patient refers to any person whose health condition is being monitored. Non-limiting examples of context data include measurement data (e.g., information about measurements of physical/physiological aspects of a subject), mental condition data (e.g., information about a psychological/psychiatric diagnosis), medical test results, geolocation information, video recordings, audio recordings, and text input.

To illustrate, when the subject is a patient, context data may include a body weight measurement, a heart rate measurement, a glucose level measurement, a water consumption measurement, a medication consumption measurement, an image, a voice input, a text input, and/or any other information that can be used to determine the health condition (e.g., physical/physiological condition and/or mental condition) of the patient. When the subject is a vehicle, context data may include geolocation information, battery level information, airbag deployment information, tire inflation information, acceleration information, an audio recording, a video recording, and/or any other information that can be used to determine the physical condition of the vehicle.

Context data source 100 may include one or more computing devices configured to store context data. For example, context data source 100 may include a database server computer system used by a hospital to store electronic health records and/or electronic medical records.

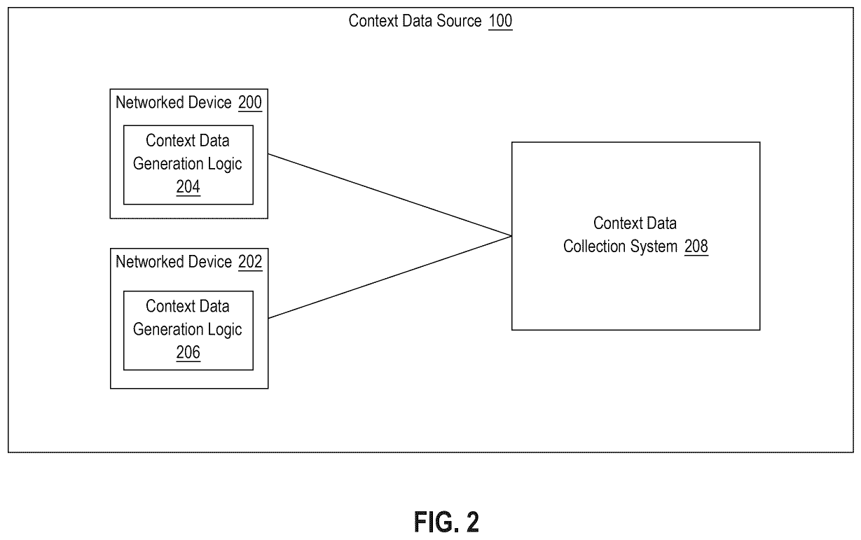

In some embodiments, context data source 100 may also include one or more computing devices configured to collect context data generated at one or more other devices. FIG. 2 depicts such an embodiment. In the example of FIG. 2, context data source 100 includes context data collection system 208 and networked devices 200-202. Context data collection system 208 is communicatively coupled to networked devices 200-202.

However, in some other embodiments, context data source 100 may include context data collection system 208 but may exclude networked devices 200-202. For example, context data collection system 208 may be a computer system used by a hospital to create or update electronic health records and/or electronic medical records based on collecting information about human-computer interactions with healthcare personnel (as opposed to inter-device communications with networked devices monitoring a patient). These records include context data such that when context data source 100 detects a change in a patient's records, an indication of the change (e.g., information about the change and/or the context data associated with the change) can be communicated from context data source 100 to notification system 102.

Referring back to the example of FIG. 2, context data collection system 208 may be implemented as one or more computing devices configured to collect context data generated at networked devices 200-202. Each networked device 200, 202 may be configured to monitor an aspect of the subject.

To illustrate, when the subject is a patient, networked devices 200-202 may be implemented as a blood pressure monitor, a heart rate monitor, a glucose monitor, a smart watch, a smart scale, a smart water bottle, a smart pill box, a network-enabled camera, a network-enabled microphone, a smartphone, a tablet computing device, an Internet of Things (IoT) device, and/or any other patient monitoring device. When the subject is a vehicle, networked devices 200-202 may be implemented as a tire pressure sensor, a global positioning system (GPS), a battery voltage detector, an accelerometer, an airbag sensor, a camera, a microphone, and/or any other vehicle monitoring device.

It should be appreciated that the foregoing lists of networked devices are not exhaustive. A networked device can be any of a myriad of possible devices for monitoring a subject, and the particular devices used may vary from implementation to implementation. Although only two networked devices are shown in the example of FIG. 2, it should be appreciated that context data collection system 208 may be communicatively coupled to any number of networked devices (including only one networked device).

In FIG. 2, networked devices 200-202 include context data generation logic 204-206, respectively. In some embodiments, context data generation logic 204, 206 may be implemented as software or firmware provided by context data collection system 208 for communicating with context data collection system 208. For example, context data generation logic 204, 206 may be provided as part of a client application for communicating with a server of context data collection system 208.

Context data generation logic 204, 206 may include instructions for determining how often networked device 200, 202 is to communicate context data to context data collection system 208. Context data can be communicated at a variety of different frequencies. Generally speaking, however, reducing the frequency of data transmission is beneficial for reducing network latency and otherwise conserving computing resources.

In some embodiments, the instructions may cause some networked devices to generate and communicate context data at regular intervals (e.g., every five seconds or every minute). For example, heart rate data may be generated every minute, and all heart rate data may be communicated to the context data collection system 208.

In some embodiments, the instructions may cause some networked devices to communicate context data only when there is a change detected in the context data. For example, blood pressure data may be communicated to the context data collection system 208 only when there is a deviation from a static reference level (e.g., 120/80 mmHg).

In some embodiments, the instructions may cause some networked devices to communicate context data only when an unexpected change is detected in the context data. For example, glucose data may be communicated to the context data collection system 208 only when there is a deviation from a dynamic reference level (e.g., 80 mg/dL in the early morning, 100 mg/dL in the late morning, 130 mg/dL in the early afternoon, 90 mg/dL in the late afternoon, and 140 mg/dL in the early evening). The dynamic reference level may change according to a digital twin model trained based on applying machine learning techniques to historical context data of the subject.

### B. Notification System

Referring back to FIG. 1, notification system 102 receives context data from context data source 100. Notification system 102 may include one or more computing devices configured to generate notifications and communicate them over a network toward recipient devices 104, 106.

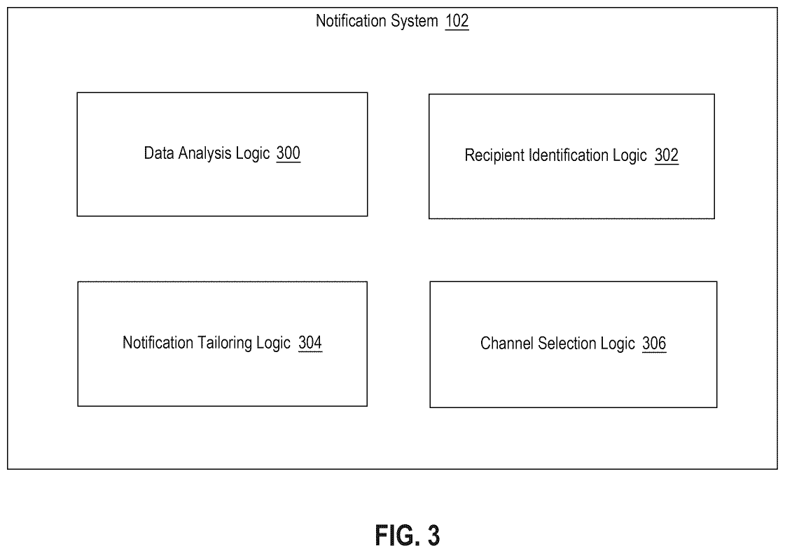

FIG. 3 depicts an example notification system. In the example of FIG. 3, notification system 102 includes data analysis logic 300, recipient selection logic 302, notification tailoring logic 304, and channel selection logic 306. Although FIG. 3 depicts notification system 102 as including logics 300-306, it should be appreciated that in some embodiments, notification system 102 may include additional logics or fewer logics. For example, in some embodiments, notification system 102 may not include notification tailoring logic 304.

Data analysis logic 300 may include instructions for performing the following:
detecting a change in the subject's condition based on analyzing context data; and/or
determining whether or not the detected change warrants generating a notification.
   In some embodiments, data analysis logic 300 may be distributed across context data source 100 and notification system 102. For example, context data source 100 may detect a change in a physical condition of a subject, and notification system 102 may determine whether or not to generate a notification based on the detected change.

Detecting a change in condition may involve performing a comparison between contemporaneously received context data and one or more reference data (e.g., previously received context data and/or a static/dynamic baseline level) to determine a difference between the compared data. In some embodiments, performing the comparison may involve applying artificial intelligence techniques (e.g., machine learning techniques, multimodal generative artificial intelligence techniques, and/or natural language processing techniques) to the contemporaneously received context data and the one or more reference data.

For example, notification system 102 may receive voice input from a patient via context data source 100. Notification system 102 may perform a comparison between the voice input and a reference voice sample taken from the patient when the patient was in a relaxed state. Performing the comparison may involve applying natural language processing techniques to the voice input and the reference voice sample. Based on analyzing differences in pitch, intonation, volume, speech rate, and/or some other paralinguistic aspect of speech, the natural language processing techniques enable detecting a change in a mental condition (e.g., a change in emotional state and/or a change in stress level) of the patient.

Determining whether or not a notification is warranted may involve performing a comparison between the detected change in condition and the subject's history of changes in condition. This comparison enables determining whether or not the detected change in condition is “normal” (e.g., consistent with an expected pattern) for the subject. In some embodiments, performing the comparison may involve providing information about the detected change as input to a machine learning model trained on information about the subject's history of changes.

For example, context data source 100 and/or notification system 102 may detect a change in a patient's physical/physiological condition based on analyzing context data indicating the patient's heart rate reached 140 bpm at 8:01 p.m. on a Tuesday. Notification system 102 may apply a machine learning model (trained on information about the patient's history of changes in heart rate) to information about the detected change in heart rate. Doing so enables notification system 102 to determine that the detected change in heart rate is not unusual for the patient at 8:01 p.m. on a Tuesday (e.g., due to a regularly scheduled exercise class). Thus, notification system 102 may determine that the detected change does not warrant generating a notification.

In some embodiments, determining whether or not a notification is warranted may further involve collecting additional context data. In such embodiments, one or more suspected causes may be determined for the detected change in condition, and upon identifying additional context data for confirming or eliminating a suspected cause, the additional context data may be collected. Based on analyzing the additional context data, a determination may be made as to whether or not to generate a notification.

For example, context data source 100 and/or notification system 102 may detect a change in a patient's physical/physiological condition based on analyzing context data indicating that the patient's glucose level is 100 mg/dL in the early afternoon. Notification system 102 may apply a machine learning model (trained on information about the patient's history of changes in glucose level) to information about the detected change in glucose level. By doing so, notification system 102 may determine that the detected change in glucose level is unusual for the patient in the early afternoon.

Continuing with the previous example, based on the patient's history of changes in glucose level, notification system 102 may also determine that skipping a meal is a suspected cause for the detected change in glucose level. Furthermore, notification system 102 may determine that skipping a meal can be confirmed or eliminated as a suspected cause by analyzing the patient's response to being asked if the patient has eaten lunch. Thus, notification system 102 may request that context data source 100 ask if the patient has had lunch (e.g., based on using a language model to generate a question prompt to be presented to the patient by a smartphone, a tablet computer, or some other networked device). Notification system 102 may also request that context data source 100 collect additional context data in the form of voice input from the patient confirming or denying that a meal was skipped.

If the voice input confirms that a meal was skipped (e.g., based on analyzing the voice input using natural language processing techniques), notification system 102 may determine that the detected change does not warrant generating a notification. However, if the voice input denies that a meal was skipped, notification system 102 may determine that a notification is warranted.

Recipient identification logic 302 may include instructions for identifying one or more recipients to be notified of the detected change in condition. A recipient may refer to a person (e.g., a spouse, a friend, a nurse, a primary care physician, or a specialist physician) or an organization of people (e.g., 911 emergency response or a station of nurses).

In some embodiments, the one or more recipients may be selected from a predetermined set of candidate recipients (e.g., a set of candidate recipients having an established relationship with the patient). Recipient selection may involve determining one or more appropriate recipients to notify under the circumstances. A recipient may be determined to be appropriate based on the context data and/or the recipient's qualifications for responding to the detected change in condition.

To illustrate, when the subject is a patient exhibiting an irregular change in heart rate at home, notification system 102 may determine that an emergency medical technician (EMT) is an appropriate recipient to notify, but when the subject is a patient exhibiting an irregular change in heart rate at a hospital, notification system 102 may determine that a nurse is an appropriate recipient to notify. When the subject is a vehicle that has deployed its airbags, notification system 102 may determine that both an EMT and a tow truck operator are appropriate recipients to notify.

In some embodiments, the cost of notifying a particular recipient may be a factor in determining whether or not identifying or selecting the recipient is appropriate. For example, when the subject is a patient exhibiting a decrease in glucose level, notification system 102 may determine that the patient's spouse is an appropriate recipient to notify and that an EMT is not an appropriate recipient to notify, because the cost (e.g., as represented by a monetary amount) of the EMT responding would be higher than the cost of the spouse responding although both the EMT and the spouse are equally qualified to respond to the detected decrease in glucose level.

In some embodiments, an artificial intelligence model (e.g., a machine learning model) may be used to identify one or more recipients to be notified of the detected change in condition. For example, the artificial intelligence model may be trained on changes in context data, qualifications for responding to changes in context data, and costs of candidate recipients. Thus, notification system 102 may apply the artificial intelligence model to the detected change in condition to generate an output indicating one or more recipients to be notified.

In some embodiments, a predetermined set of rules may be used to identify one or more recipients to be notified of the detected change in condition. For example, the predetermined set of rules may specify that an EMT is to be notified if the patient exhibits an irregular change in heart rate at home, that a nurse be notified if the patient exhibits an irregular change in heart rate at a hospital, that the patient's spouse be notified if the patient exhibits a decrease in glucose level at home, etc.

Advantageously, using a predetermined set of rules in conjunction with an artificial intelligence model can reduce or eliminate incidents of hallucination that can occur by using the artificial intelligence model alone. To use a predetermined set of rules in conjunction with an artificial intelligence model, the artificial intelligence model may be trained on the predetermined set of rules in addition to changes in context data, qualifications for responding to changes in context data, and costs of candidate recipients. Additionally or alternatively, the predetermined set of rules may be provided as input to the artificial intelligence model.

In some embodiments, if none of the candidate recipients in the predetermined set of candidate recipients is qualified to respond to the detected change in condition, then a recipient may be identified from outside the predetermined set of candidate recipients. For example, when a patient exhibits a change in condition that is of a type previously unexhibited by the patient (e.g., depression detected in a patient with no history of depression), notification system 102 may identify a recipient without an established relationship with the patient (e.g., a psychiatrist). Thus, to comply with the Health Insurance Portability and Accountability Act (HIPAA), notification system 102 may request permission from the patient (or an authorized proxy of the patient) to notify the recipient without an established relationship with the patient.

Notification tailoring logic 304 may include instructions for customizing notification content according to the intended recipient. More specifically, context data may be tailored to the recipient's qualifications for responding to a change in the subject's condition. In some embodiments, tailored context data may be generated based on applying a language model to the context data and the recipient's qualifications for responding to the change.

For example, notification system 102 may determine that a patient's spouse is to be notified that the patient's blood pressure has reached 130/90 mmHg. Since the patient's spouse has no medical training, instead of generating a notification including the numerical value of the patient's blood pressure measurement, notification system 102 may generate a notification including an interpretation of the patient's blood pressure measurement (e.g., a comment that the patient's blood pressure is elevated) and/or a recommendation for restoring the patient's blood pressure to a normal level (e.g., a suggestion to perform a soothing activity for the patient).

Channel selection logic 306 may include instructions for selecting an appropriate communication channel for notifying a recipient. In some embodiments, the appropriate communication channel may be selected from a plurality of candidate communication channels (e.g., mobile phone, office phone, Short Message Service, electronic mail, and regular mail). In some embodiments, the appropriate communication channel may be selected based on the urgency of eliciting a response to a change in the subject's condition. A predetermined set of rules may be used to select the appropriate communication channel for a particular situation.

For example, notification system 102 may determine that both a nurse and a doctor are to be notified of an increase in a patient's glucose level. Notification system 102 may communicate a notification toward the nurse via Short Message Service, whereas notification system 102 may communicate a notification toward the doctor via electronic mail.

### C. Recipient Device(s)

Referring back to FIG. 1, recipient devices 104-106 receive notifications generated by notification system 102. A recipient device may be any computing device that can be used to receive a notification on behalf of an intended recipient (e.g., a person and/or an organization of people) of the notification. Non-limiting examples of recipient devices include smartphones, tablet computers, desktop computers, personal computers, workstations, and/or smart televisions. Although only two recipient devices are shown in the example of FIG. 1, it should be appreciated that notification system 102 may be communicatively coupled to any number of recipient devices (including only one recipient device).

In some embodiments, a single recipient device may be used to receive notifications on behalf of multiple recipients. For example, a smart television may be used to receive a notification intended for any of the nurses who work in the area of the hospital where the smart television is located.

In some embodiments, multiple recipient devices may be used to receive notifications on behalf of a single recipient. For example, a nurse may be notified via a smartphone, a workstation computer, and/or a smart television.

### III. Approaches for Intelligent Notification Delivery

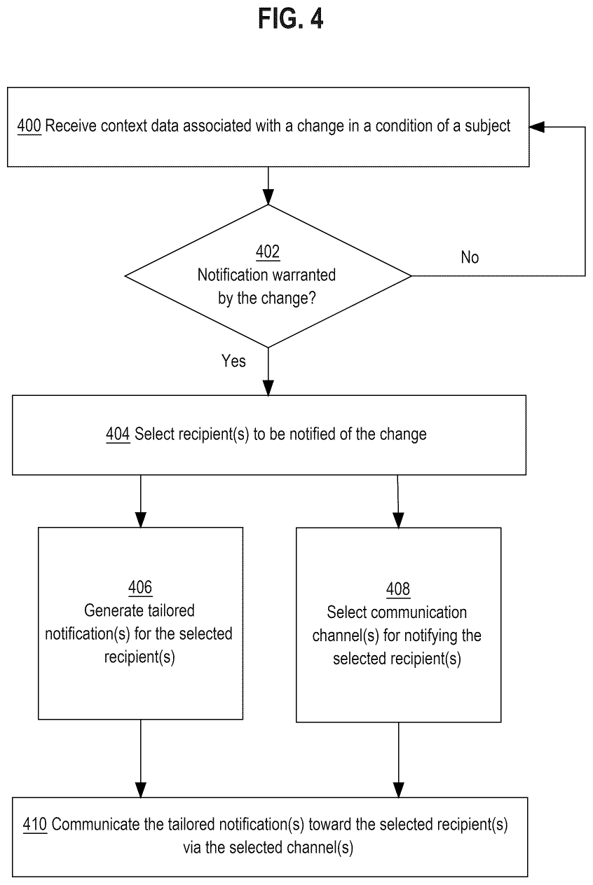

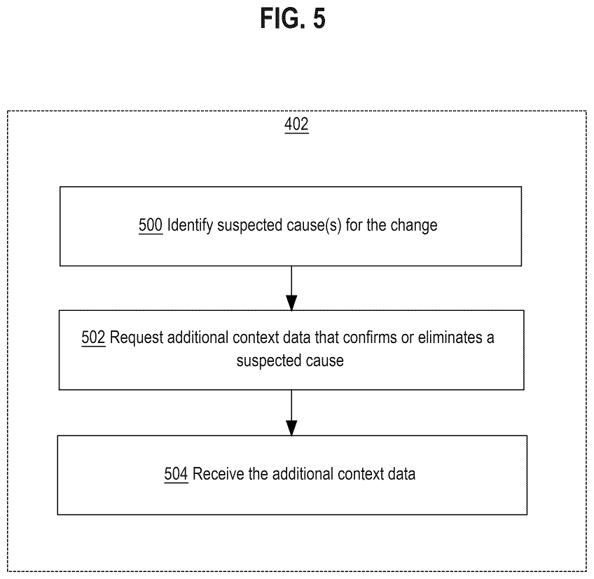

FIGS. 4-5 are flow diagrams that depict example approaches for performing intelligent notification delivery, according to embodiments. Although the flow diagrams depict blocks in a particular order, some other implementations may use fewer or more blocks, in the same or different order.

For the sake of clarity, examples are provided in the context of patient monitoring. However, it should be appreciated that the approaches described herein are equally applicable to other contexts (e.g., vehicle monitoring).

At block 400 of FIG. 4, context data is received. For example, notification system 102 may receive context data including measurement data and/or mental condition data from context data source 100.

The context data is associated with a change in a condition of a subject. In some embodiments, the context data may be associated with a change in a health condition of a patient. For example, the context data may include measurement data (e.g., a glucose level measurement) which, when compared to reference data (e.g., a fasting glucose level), indicates a change in health condition (e.g., hyperglycemia or hypoglycemia).

In some embodiments, the context data may be generated at one or more networked devices configured to monitor the subject. In such embodiments, context data may be received from the one or more networked devices and stored at one or more central server computers. For example, context data generated at a glucose level monitor, a smartwatch, a smart scale, a smart water bottle, and a smart pillbox may be collected and stored at context data collection system 208.

In some embodiments, the context data may be obtained from electronic health records or electronic medical records of the subject. For example, the context data may be manually entered into a hospital computer system that pushes underlying context data to notification system 102 when a change is detected in the records (and, thus, in a health condition of the patient).

At block 402, a determination is made as to whether or not a notification is warranted by the change. If a determination is made that notifying a recipient of a change in the subject's condition is warranted, then block 402 proceeds to block 404. Otherwise, block 402 proceeds to block 400.

In some embodiments, the determination may be made based on whether or not a change in the subject's condition is detected. Detecting a change in the subject's condition may involve analyzing context data. For example, a change in a physical/physiological condition (e.g., a glucose level) of a patient may be detected based on analyzing measurement data (e.g., a glucose level measurement), and a change in a mental condition (e.g., depression) of the patient may be detected based on analyzing user input (e.g., applying natural language processing techniques to voice or text input) provided by the patient.

In some embodiments, determining whether or not a notification is warranted may involve analyzing additional context data. FIG. 5 depicts such an embodiment. Referring to FIG. 5, block 402 includes blocks 500-504.

At block 500, one or more suspected causes for the change are identified. For example, one or more suspected causes (e.g., a skipped meal and/or a hormonal change) for a change in physical/physiological condition (e.g., a decrease in glucose level) may be identified based on mental condition data (e.g., information suggesting that the patient is depressed) and the change in physical/physiological condition. In some embodiments, a predetermined set of rules may specify one or more suspected causes for each change in health condition that may be detected. In some embodiments, multimodal generative artificial intelligence techniques may be used to identify one or more suspected causes for a change in health condition (e.g., by generating an output indicative of one or more suspected causes based on applying a large multimodal model to a change in health condition and/or context data underlying the change in health condition).

At block 502, additional context data is requested to confirm or eliminate a suspected cause of the one or more suspected causes identified at block 500. For example, to confirm or eliminate a skipped meal as a suspected cause for a decrease in glucose level, notification system 102 may request, from context data source 100, the patient's response to being asked whether a meal was skipped.

In some embodiments, the additional context data may be requested by ordering a medical test that provides insight about the change in the condition. For example, notification system 102 may order a blood test to check the levels of various hormones. Thus, in some embodiments, the additional context data may be generated at (and, thus, ultimately received from) one or more networked devices that are different from the one or more networked devices used to generate the context data received at block 400.

In some embodiments, the additional context data may be requested by initiating establishment of a two-way communication between the subject and a potential notification recipient. For example, notification system 102 may initiate establishment of a two-way communication between the patient and a nurse so that the nurse can ask the patient if a meal was skipped.

At block 504, the additional context data is received. For example, notification system 102 may receive additional context data in the form of user input (e.g., voice input or text input) provided by the patient in response to being asked whether a meal was skipped.

The additional context data may be analyzed to determine whether or not the change in the condition of the subject warrants communicating, to one or more recipients, a notification of the change. Continuing with the previous example, the user input may confirm that a meal was skipped. Additionally, a change in a mental condition of the patient may be detected based on applying natural language techniques and/or emotion analysis to the user input. For example, the user input may include a voice recording which, when analyzed, suggests that the patient is no longer suffering from depression.

Thus, a determination may be made that the change in physical/physiological condition and/or the change in mental condition warrant communicating one or more notifications. For example, notification system 102 may determine that a nurse should be notified of the change in physical/physiological condition and that a psychiatrist should be notified of both the change in physical/physiological condition and the change in mental condition.

In some embodiments, a determination may be made to notify a recipient that does not have an established relationship with the subject. This may occur, because the recipient is more qualified to respond to the change in condition than any of the candidate recipients that do have an established relationship with the subject. In such embodiments, permission may be received from the subject or an authorized proxy of the subject (e.g., a person with power of attorney) prior to communicating, toward the recipient, the notification of the change in the condition of the subject.

Referring back to FIG. 4, at block 404, one or more recipients are selected to be notified of the change. In some embodiments, the one or more recipients may be selected from a plurality of candidate recipients. For example, the plurality of recipients may include the patient's spouse, the patient's primary care physician, the patient's gynecologist, and an emergency medical technician. In some embodiments, the one or more recipients may be selected based on applying an artificial intelligence model to the change in the condition of the subject.

Block 404 proceeds to block 406 and block 408. Although FIG. 4 depicts blocks 406-408 as being performed concurrently, it should be appreciated that block 406 can be performed before or after block 408 in some other implementations.

At block 406, one or more tailored notifications are generated for the one or more recipients selected at block 404. In some embodiments, generating a tailored notification may involve generating tailored data and including it in a notification to be communicated to a selected recipient. The tailored data may be generated based on tailoring the context data to qualifications of the selected recipient for responding to the change.

For example, the selected recipient may be the patient's spouse, who does not have a medical background. Accordingly, if the context data includes a glucose level measurement, the context data may be tailored such that the numerical value of the glucose level measurement is omitted in favor of an interpretation of glucose level measurement as low. Stated differently, the numerical value may be excluded, because it may not be readily understandable for a person having the selected recipient's qualifications for responding to the change.

In some embodiments, generating tailored data may involve applying a language model (e.g., a large language model) to the context data and the qualifications for responding to the change. Thus, the language model may be an audience-aware language model that accounts for a specified audience when editing context data.

When a plurality of tailored notifications are generated, the tailored notifications may be generated sequentially or concurrently. For example, after a first tailored notification is generated for a first recipient, a second tailored notification may be generated for a second recipient. Alternatively, the first tailored notification and the second tailored notification may be generated in parallel.

In some embodiments, notifications are tailored differently for different recipients. A first notification (including first tailored data) to be communicated to a first recipient may differ from a second notification (including second tailored data) to be communicated to a second recipient, because the first recipient and the second recipient may differ in qualifications for responding to the change. Thus, the first tailored data is different from the second tailored data.

For example, both the patient's spouse and the patient's primary care physician may be selected to be notified of a change in glucose level. In contrast to the patient's spouse, the patient's primary care physician does have a medical background. Thus, although the patient's spouse may not be notified of the numerical value of the glucose level measurement, the patient's primary care physician may be notified of this numerical value along with one or more suspected causes for the change in glucose level.

At block 408, one or more communication channels are selected for notifying the one or more recipients selected at block 404. The one or more communication channels may be selected from a plurality of candidate communication channels (e.g., phone call, video call, text message, electronic mail, “smart” app notification, and regular mail). In some embodiments, the one or more communication channels may be selected based on urgency of eliciting a response to the change.

For example, a predetermined set of rules may correlate recipients, communication channels, and changes in the health condition of the patient. The predetermined set of rules may specify that if the patient's glucose level is between 180 mg/dL and 200 mg/dL, the patient's spouse is to be notified via text message. The predetermined set of rules may also specify that if the patient's glucose level is over 200 mg/dL, the patient's spouse is to be notified via phone call and the patient's primary care physician is to be notified via text message.

At block 410, the one or more tailored notifications are communicated toward the one or more recipients selected at block 404. The one or more tailored notifications are communicated via the one or more communication channels selected at block 408. Communicating a tailored notification toward a selected recipient may involve communicating the tailored notification over a network to a computing device belonging to, used by, or otherwise associated with the selected recipient.

In some embodiments, communicating the one or more tailored notifications may be performed in conjunction with one or more additional actions. For example, notification system 102 may initiate establishment of a two-way communication between the subject and a selected recipient, may order medication that counteracts the change in the condition of the subject, and/or may order a medical test that provides insight about the change in the condition.

### IV. Implementation Mechanisms

Intelligent notification delivery as described herein leverages artificial intelligence to enable faster and more effective responses than prior approaches for notification delivery. Artificial intelligence expedites the difficult task of determining the most appropriate recipient for a notification under a myriad of different circumstances. The approach also reduces bandwidth consumption and otherwise conserves computing resources by avoiding unnecessary notifications.

Example artificial intelligence techniques and other implementation mechanisms are described below. The particular techniques and mechanisms that are employed may vary from implementation to implementation.

### Machine Learning Models

A machine learning model is trained using a particular machine learning algorithm. Once trained, input is applied to the machine learning model to make a prediction, which may also be referred to herein as a predicated output or output. Attributes of the input may be referred to as features and the values of the features may be referred to herein as feature values.

A machine learning model includes a model data representation or model artifact. A model artifact comprises parameters values, which may be referred to herein as theta values, and which are applied by a machine learning algorithm to the input to generate a predicted output. Training a machine learning model entails determining the theta values of the model artifact. The structure and organization of the theta values depend on the machine learning algorithm.

In supervised training, training data is used by a supervised training algorithm to train a machine learning model. The training data includes input and a “known” output. In an embodiment, the supervised training algorithm is an iterative procedure. In each iteration, the machine learning algorithm applies the model artifact and the input to generate a predicted output. An error or variance between the predicted output and the known output is calculated using an objective function. In effect, the output of the objective function indicates the accuracy of the machine learning model based on the particular state of the model artifact in the iteration. By applying an optimization algorithm based on the objective function, the theta values of the model artifact are adjusted. An example of an optimization algorithm is gradient descent. The iterations may be repeated until a desired accuracy is achieved or some other criteria are met.

In a software implementation, when a machine learning model is referred to as receiving an input, being executed, and/or generating an output or prediction, a computer system process executing a machine learning algorithm applies the model artifact against the input to generate a predicted output. A computer system process executes a machine learning algorithm by executing software configured to cause execution of the algorithm. When a machine learning model is referred to as performing an action, a computer system process executes a machine learning algorithm by executing software configured to cause performance of the action.

Inferencing entails a computer applying the machine learning model to an input such as a feature vector to generate an inference by processing the input and content of the machine learning model in an integrated way. Inferencing is data driven according to data, such as learned coefficients, that the machine learning model contains. Herein, this is referred to as inferencing by the machine learning model that, in practice, is execution by a computer of a machine learning algorithm that processes the machine learning model.

Classes of problems that machine learning (ML) excels at include clustering, classification, regression, anomaly detection, prediction, and dimensionality reduction (i.e. simplification). Examples of machine learning algorithms include decision trees, support vector machines (SVM), Bayesian networks, stochastic algorithms such as genetic algorithms (GA), and connectionist topologies such as artificial neural networks (ANN). Implementations of machine learning may rely on matrices, symbolic models, and hierarchical and/or associative data structures. Parameterized (i.e. configurable) implementations of the best breed machine learning algorithms may be found in open source libraries such as Google's TensorFlow for Python and C++ or Georgia Institute of Technology's MLPack for C++. Shogun is an open source C++ ML library with adapters for several programing languages including C #, Ruby, Lua, Java, MatLab, R, and Python.

### Artificial Neural Networks

An artificial neural network (ANN) is a machine learning model that at a high level models a system of neurons interconnected by directed edges. An overview of neural networks is described within the context of a layered feedforward neural network. Other types of neural networks share characteristics of neural networks described below.

In a layered feed forward network, such as a multilayer perceptron (MLP), each layer comprises a group of neurons. A layered neural network comprises an input layer, an output layer, and one or more intermediate layers referred to hidden layers.

Neurons in the input layer and output layer are referred to as input neurons and output neurons, respectively. A neuron in a hidden layer or output layer may be referred to herein as an activation neuron. An activation neuron is associated with an activation function. The input layer does not contain any activation neurons.

From each neuron in the input layer and a hidden layer, there may be one or more directed edges to an activation neuron in the subsequent hidden layer or output layer. Each edge is associated with a weight. An edge from a neuron to an activation neuron represents input from the neuron to the activation neuron, as adjusted by the weight.

For a given input to a neural network, each neuron in the neural network has an activation value. For an input neuron, the activation value is simply an input value for the input. For an activation neuron, the activation value is the output of the respective activation function of the activation neuron.

Each edge from a particular neuron to an activation neuron represents that the activation value of the particular neuron is an input to the activation neuron, that is, an input to the activation function of the activation neuron, as adjusted by the weight of the edge. Thus, an activation neuron in the subsequent layer represents that the particular neuron's activation value is an input to the activation neuron's activation function, as adjusted by the weight of the edge. An activation neuron can have multiple edges directed to the activation neuron, each edge representing that the activation value from the originating neuron, as adjusted by the weight of the edge, is an input to the activation function of the activation neuron.

Each activation neuron is associated with a bias. To generate the activation value of an activation neuron, the activation function of the neuron is applied to the weighted activation values and the bias.

### Illustrative Data Structures for Neural Network

The artifact of a neural network may comprise matrices of weights and biases. Training a neural network may iteratively adjust the matrices of weights and biases.

For a layered feedforward network, as well as other types of neural networks, the artifact may comprise one or more matrices of edges W. A matrix W represents edges from a layer L−1 to a layer L. Given the number of neurons in layer L−1 and L is N[L−1] and N[L], respectively, the dimensions of matrix W is N[L−1] columns and N[L] rows.

Biases for a particular layer L may also be stored in matrix B having one column with N[L] rows.

The matrices W and B may be stored as a vector or an array in RAM memory, or comma separated set of values in memory. When an artifact is persisted in persistent storage, the matrices W and B may be stored as comma separated values, in compressed and/serialized form, or other suitable persistent form.

A particular input applied to a neural network comprises a value for each input neuron. The particular input may be stored as a vector. Training data comprises multiple inputs, each being referred to as a sample in a set of samples. Each sample includes a value for each input neuron. A sample may be stored as a vector of input values, while multiple samples may be stored as a matrix, each row in the matrix being a sample.

When an input is applied to a neural network, activation values are generated for the hidden layers and output layer. For each layer, the activation values for may be stored in one column of a matrix A having a row for every neuron in the layer. In a vectorized approach for training, activation values may be stored in a matrix, having a column for every sample in the training data.

Training a neural network requires storing and processing additional matrices. Optimization algorithms generate matrices of derivative values which are used to adjust matrices of weights W and biases B. Generating derivative values may use and require storing matrices of intermediate values generated when computing activation values for each layer.

The number of neurons and/or edges determines the size of matrices needed to implement a neural network. The smaller the number of neurons and edges in a neural network, the smaller matrices and amount of memory needed to store matrices. In addition, a smaller number of neurons and edges reduces the amount of computation needed to apply or train a neural network. Fewer neurons means fewer activation values need be computed, and/or fewer derivative values need be computed during training.

Properties of matrices used to implement a neural network correspond to neurons and edges. A cell in a matrix W represents a particular edge from a neuron in layer L−1 to L. An activation neuron represents an activation function for the layer that includes the activation function. An activation neuron in layer L corresponds to a row of weights in a matrix W for the edges between layer L and L−1 and a column of weights in a matrix W for edges between layer L and L+1. During execution of a neural network, a neuron also corresponds to one or more activation values stored in matrix A for the layer and generated by an activation function.

An ANN is amenable to vectorization for data parallelism, which may exploit vector hardware such as single instruction multiple data (SIMD), such as with a graphical processing unit (GPU). Matrix partitioning may achieve horizontal scaling such as with symmetric multiprocessing (SMP) such as with a multicore central processing unit (CPU) and or multiple coprocessors such as GPUs. Feed forward computation within an ANN may occur with one step per neural layer. Activation values in one layer are calculated based on weighted propagations of activation values of the previous layer, such that values are calculated for each subsequent layer in sequence, such as with respective iterations of a for loop. Layering imposes sequencing of calculations that are not parallelizable. Thus, network depth (i.e. amount of layers) may cause computational latency. Deep learning entails endowing a multilayer perceptron (MLP) with many layers. Each layer achieves data abstraction, with complicated (i.e. multidimensional as with several inputs) abstractions needing multiple layers that achieve cascaded processing. Reusable matrix-based implementations of an ANN and matrix operations for feed forward processing are readily available and parallelizable in neural network libraries such as Google's TensorFlow for Python and C++, OpenNN for C++, and University of Copenhagen's fast artificial neural network (FANN). These libraries also provide model training algorithms such as backpropagation.

### Backpropagation

An ANN's output may be more or less correct. For example, an ANN that recognizes letters may mistake an I as an L because those letters have similar features. Correct output may have particular value(s), while actual output may have somewhat different values. The arithmetic or geometric difference between correct and actual outputs may be measured as error according to a loss function, such that zero represents error free (i.e. completely accurate) behavior. For any edge in any layer, the difference between correct and actual outputs is a delta value.

Backpropagation entails distributing the error backward through the layers of the ANN in varying amounts to all of the connection edges within the ANN. Propagation of error causes adjustments to edge weights, which depend on the gradient of the error at each edge. Gradient of an edge is calculated by multiplying the edge's error delta times the activation value of the upstream neuron. When the gradient is negative, the greater the magnitude of error contributed to the network by an edge, the more the edge's weight should be reduced, which is negative reinforcement. When the gradient is positive, then positive reinforcement entails increasing the weight of an edge whose activation reduced the error. An edge weight is adjusted according to a percentage of the edge's gradient. The steeper is the gradient, the bigger is adjustment. Not all edge weights are adjusted by a same amount. As model training continues with additional input samples, the error of the ANN should decline. Training may cease when the error stabilizes (i.e. ceases to reduce) or vanishes beneath a threshold (i.e. approaches zero). Example mathematical formulae and techniques for feedforward multilayer perceptron (MLP), including matrix operations and backpropagation, are taught in related reference “EXACT CALCULATION OF THE HESSIAN MATRIX FOR THE MULTI-LAYER PERCEPTRON,” by Christopher M. Bishop.

Model training may be supervised or unsupervised. For supervised training, the desired (i.e. correct) output is already known for each example in a training set. The training set is configured in advance by (e.g. a human expert) assigning a categorization label to each example. For example, the training set for optical character recognition may have blurry photographs of individual letters, and an expert may label each photo in advance according to which letter is shown. Error calculation and backpropagation occur as explained above.

### Autoencoder

Unsupervised model training is more involved because desired outputs need to be discovered during training. Unsupervised training may be easier to adopt because a human expert is not needed to label training examples in advance. Thus, unsupervised training saves human labor. A natural way to achieve unsupervised training is with an autoencoder, which is a kind of ANN. An autoencoder functions as an encoder/decoder (codec) that has two sets of layers. The first set of layers encodes an input example into a condensed code that needs to be learned during model training. The second set of layers decodes the condensed code to regenerate the original input example. Both sets of layers are trained together as one combined ANN. Error is defined as the difference between the original input and the regenerated input as decoded. After sufficient training, the decoder outputs more or less exactly whatever is the original input.

An autoencoder relies on the condensed code as an intermediate format for each input example. It may be counter-intuitive that the intermediate condensed codes do not initially exist and instead emerge only through model training. Unsupervised training may achieve a vocabulary of intermediate encodings based on features and distinctions of unexpected relevance. For example, which examples and which labels are used during supervised training may depend on somewhat unscientific (e.g. anecdotal) or otherwise incomplete understanding of a problem space by a human expert. Whereas unsupervised training discovers an apt intermediate vocabulary based more or less entirely on statistical tendencies that reliably converge upon optimality with sufficient training due to the internal feedback by regenerated decodings. Techniques for unsupervised training of an autoencoder for anomaly detection based on reconstruction error is taught in non-patent literature (NPL) “VARIATIONAL AUTOENCODER BASED ANOMALY DETECTION USING RECONSTRUCTION PROBABILITY”, Special Lecture on IE. 2015 Dec. 27; 2(1): 1-18 by Jinwon An et al.

### Principal Component Analysis

Principal component analysis (PCA) provides dimensionality reduction by leveraging and organizing mathematical correlation techniques such as normalization, covariance, eigenvectors, and eigenvalues. PCA incorporates aspects of feature selection by eliminating redundant features. PCA can be used for prediction. PCA can be used in conjunction with other ML algorithms.

### Random Forest

A random forest or random decision forest is an ensemble of learning approaches that construct a collection of randomly generated nodes and decision trees during a training phase. Different decision trees of a forest are constructed to be each randomly restricted to only particular subsets of feature dimensions of the data set, such as with feature bootstrap aggregating (bagging). Therefore, the decision trees gain accuracy as the decision trees grow without being forced to over fit training data as would happen if the decision trees were forced to learn all feature dimensions of the data set. A prediction may be calculated based on a mean (or other integration such as soft max) of the predictions from the different decision trees.

Random forest hyper-parameters may include: number-of-trees-in-the-forest, maximum-number-of-features-considered-for-splitting-a-node, number-of-levels-in-each-decision-tree, minimum-number-of-data-points-on-a-leaf-node, method-for-sampling-data-points, etc.

### Generative Models

A generative model is a machine learning model that is capable of generating new data instances based on the data used to train the model. A generative model may be referred to as a “generative artificial intelligence (AI) model.” Generative models learn the underlying distribution of the training data, enabling them to produce new instances of data that share properties with the original dataset. This capability makes them particularly useful in a variety of applications, including image and voice generation, text synthesis, and more sophisticated tasks like unsupervised learning, semi-supervised learning, and domain adaptation.

One type of generative model is a large language model. Large language models are designed to understand, generate, and interpret human language by processing extensive collections of data. The foundational architecture behind large language models is the transformer network, a type of neural network that excels in handling sequential data such as text. Unlike architectures, such as recurrent neural networks (RNNs) or long short-term memory networks (LSTMs), transformers do not process data in order. Instead, they leverage parallel processing to analyze entire text sequences simultaneously, significantly improving efficiency and reducing training times.

In an embodiment, a mechanism that enables transformers to handle complex language tasks is self-attention. This mechanism allows the model to weigh the importance of different words within a sentence or sequence regardless of their position. For instance, in processing the phrase “The cat sat on the mat,” the model can directly associate “cat” with “mat” without having to process the intermediate words sequentially. This ability to understand the context and relationships between words in a sentence is what makes transformer networks adept at language tasks. The self-attention mechanism assigns scores to relationships between words, highlighting the most relevant connections, so the model can focus on the most informative parts of the text.

In accordance with one or more embodiments, transformers are composed of multiple layers containing a multi-head, self-attention mechanism and a position-wise, feed-forward network. Within the architecture of transformer models, the multi-head, self-attention mechanism and position-wise, feed-forward network function in concert to process input data. The multi-head, self-attention mechanism is designed to enable parallel processing of input sequences, allowing the model to simultaneously evaluate the importance of different segments of the input relative to each other. This mechanism operates by generating multiple sets of query, key, and value vectors for each element in the input sequence through linear transformation. The relevance of each element to every other element is calculated using a scaled dot-product attention function that computes the attention scores by taking the dot product of the query vector with the key vectors, dividing each by the square root of the dimension of the key vectors to scale the scores, then applying a softmax function to obtain the weights for the value vectors. The scaled dot-product attention function is applied independently by each head in the multi-head self-attention mechanism. The outputs of these heads are then concatenated and linearly transformed, allowing the model to capture information from different representation subspaces.

In accordance with one or more embodiments, following the multi-head, self-attention mechanism is the position-wise, feed-forward network. This component comprises two linear transformations with a non-linear activation function in between. Each element of the input sequence, now enriched with context by the self-attention mechanism, is processed independently through the same feed-forward network. The first linear transformation increases the dimensionality of the input, allowing for a richer representation space. The non-linear activation function introduces the capability to capture non-linear relationships within the data. The second linear transformation then reduces the dimensionality back to that of the model's hidden layers, preparing the output for either further processing by subsequent layers or final output generation. This sequence of operations is applied to each position in the sequence, so the model can learn complex patterns across different parts of the input data without relying on the sequential processing inherent to previous architectures, such as RNNs or LSTMs.

In accordance with one or more embodiments, integrating these components within the transformer architecture facilitates the model's ability to understand and generate human language by leveraging both the global context provided by the self-attention mechanism and the local, position-specific transformations applied by the feed-forward networks. Through the repetitive stacking of layers, transformers achieve a depth of representation that allows for the processing of linguistic information across varying levels of complexity.

Another type of generative model is a large multimodal model (LMM). A large multimodal model is an advanced machine learning model capable of processing and generating data across multiple modalities, such as text, images, audio, and video. These models integrate diverse datasets during training to learn the underlying distribution of different data types, enabling them to produce outputs that reflect a comprehensive understanding of the input data. These models can be used for applications such as image captioning, text-to-image generation, image-to-text generation, visual question answering, and more, where understanding the relationship between different data types is crucial. By leveraging diverse datasets during training, large multimodal models learn to create coherent and contextually relevant outputs across various modalities, enhancing their utility in complex, real-world scenarios.

The architecture of large multimodal models combines elements from different neural network designs to handle diverse data types effectively. For example, convolutional neural networks (CNNs) are often used for processing visual data, while transformer networks handle textual data, enabling the model to extract and synthesize features from both images and text. This integration results in outputs that accurately represent the input data, reflecting a deep understanding of both modalities. The transformer architecture, known for its ability to manage sequential data, is frequently adapted to work alongside CNNs, allowing these models to benefit from the strengths of each neural network type.

In at least some instances, the self-attention mechanism, a cornerstone of transformer networks, is integral to the functioning of large multimodal models. It enables the model to weigh the importance of different elements within an input sequence, regardless of their position, allowing it to capture intricate relationships between various data types. For example, in an image captioning task, the model can associate specific visual features with corresponding descriptive text, enhancing the coherence and accuracy of the generated captions. By assigning scores to relationships between elements, the self-attention mechanism highlights the most relevant connections, enabling the model to focus on the most informative parts of the input data and perform complex multimodal tasks effectively.

In large multimodal models, data preprocessing ensures the input data is in a suitable format for the model to process. This involves tasks such as tokenization for text data, where the text is broken down into manageable pieces, and feature extraction for image data, where key visual elements are identified and encoded. By standardizing and normalizing different data types, preprocessing reduces the complexity of the input space, enabling the model to treat similar elements consistently. Effective preprocessing is essential for the model to integrate information from various modalities and produce accurate, meaningful outputs.

Training large multimodal models involves optimizing their parameters through exposure to diverse datasets that include paired data from different modalities. This computationally intensive process often requires specialized hardware like GPUs or TPUs to manage the large volumes of data and the complexity of the model calculations. Techniques such as dropout and layer normalization are employed to improve model generalization and prevent overfitting. By iteratively adjusting the model's parameters, the training process enables the model to learn underlying patterns and relationships within the data, enhancing its ability to generate coherent and contextually relevant outputs across different modalities.

Evaluation and tuning of large multimodal models are conducted using various metrics tailored to the specific tasks they are designed to perform. For example, BLEU scores are used for text generation tasks, while accuracy is commonly applied for visual recognition tasks to assess performance. Tuning involves adjusting hyperparameters and refining training strategies based on evaluation results to enhance the model's effectiveness. This iterative process ensures that the model can perform a wide range of multimodal tasks with high accuracy and relevance, making it a versatile tool for applications requiring the integration of different types of data.

Large multimodal models represent a significant advancement in machine learning by leveraging sophisticated architectures that combine different neural network types and apply self-attention mechanisms. This enables them to perform complex tasks that require understanding and synthesizing information from diverse data types. Effective preprocessing, rigorous training, and thorough evaluation are crucial to their success, allowing these models to generate coherent and contextually relevant outputs across a wide range of applications.

In accordance with one or more embodiments, other types of models besides large language models and large multimodal models belong to the broad category of generative models. For example, stochastic models directly incorporate randomness into their structure, making them inherently generative as they can produce a diverse set of outputs for a given input. Generative Adversarial Networks (GANs) learn to generate new data that is indistinguishable from the data they were trained on, using a dual-network architecture that involves a generative component. Variational Autoencoders (VAEs) are explicitly designed for generating new data points by learning a distribution of the input data and encode inputs into a latent space and generate outputs by sampling from this space, making them inherently generative. Sequence-to-sequence models are generative in nature when used with sampling strategies. Although this list of generative model types is not exhaustive, it illustrates the broad use of the term generative model beyond large language models.

Although generative models can be leveraged for classification tasks, they inherently operate on principles of randomness, leading to a spectrum of possible outcomes in response to identical inputs. Unlike deterministic models that yield a consistent result whenever the same input is given, generative models use the randomness in the data they are trained on to both mimic and diversify from the training data. This diversity makes generative models ideal for generating new and varied data points as well as for tasks that require creativity and novelty. However, a reliance on randomness creates a trade-off between predictability and flexibility for generative models, potentially making them less predictable in scenarios where uniform outcomes may be expected such as classification tasks.

### Software Overview

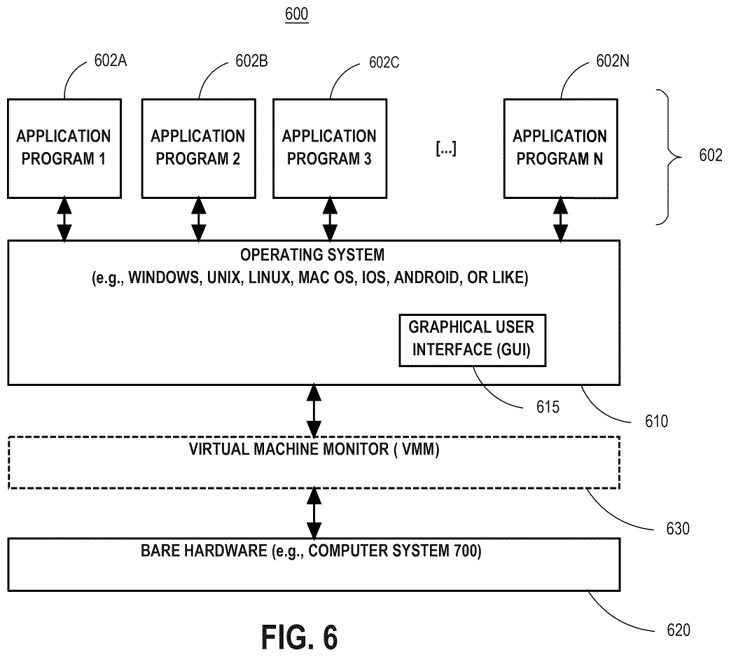

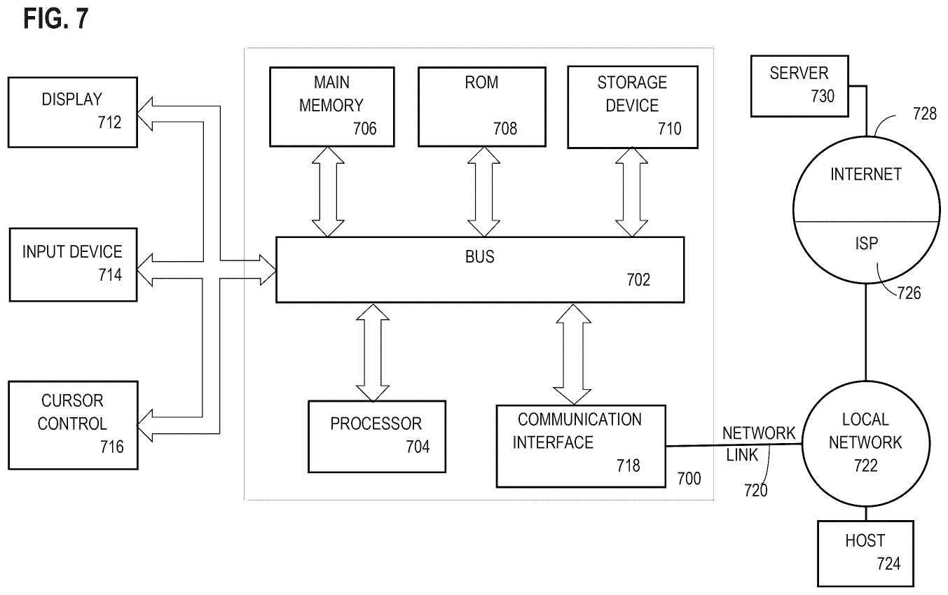

FIG. 6 is a block diagram of a basic software system 600 that may be employed for controlling the operation of computing system 700 of FIG. 7. Software system 600 and its components, including their connections, relationships, and functions, are meant to be exemplary only and not meant to limit implementations of the example embodiment(s). Other software systems suitable for implementing the example embodiment(s) may have different components, including components with different connections, relationships, and functions.

Software system 600 is provided for directing the operation of computing system 700. Software system 600, which may be stored in system memory (RAM) 706 and on fixed storage (e.g., hard disk or flash memory) 710, includes a kernel or operating system (OS) 610.

The OS 610 manages low-level aspects of computer operation, including managing the execution of processes, memory allocation, file input and output (I/O), and device I/O. One or more application programs represented as 602A, 602B, 602C . . . 602N may be “loaded” (e.g., transferred from fixed storage 710 into memory 706) for execution by the system 600. The applications or other software intended for use on computer system 700 may also be stored as a set of downloadable computer-executable instructions, for example, for downloading and installation from an Internet location (e.g., a Web server, an app store, or another online service).

Software system 600 includes a graphical user interface (GUI) 615 for receiving user commands and data in a graphical (e.g., “point-and-click” or “touch gesture”) fashion. These inputs, in turn, may be acted upon by system 600 in accordance with instructions from operating system 610 and/or application(s) 602. The GUI 615 also serves to display the results of operation from OS 610 and application(s) 602, whereupon the user may supply additional inputs or terminate the session (e.g., log off).

OS 610 can execute directly on the bare hardware 620 (e.g., processor(s) 704) of computer system 700. Alternatively, a hypervisor or virtual machine monitor (VMM) 630 may be interposed between the bare hardware 620 and the OS 610. In this configuration, VMM 630 acts as a software “cushion” or virtualization layer between OS 610 and the bare hardware 620 of the computer system 700.

VMM 630 instantiates and runs one or more virtual machine instances (“guest machines”). Each guest machine comprises a “guest” operating system, such as OS 610, and one or more applications, such as application(s) 602, designed to execute on the guest operating system. The VMM 630 presents the guest operating systems with a virtual operating platform and manages the execution of the guest operating systems.

In some instances, the VMM 630 may allow a guest operating system to run as if it is running on the bare hardware 620 of computer system 700 directly. In these instances, the same version of the guest operating system configured to execute on the bare hardware 620 directly may also execute on VMM 630 without modification or reconfiguration. In other words, VMM 630 may provide full hardware and CPU virtualization to a guest operating system in some instances.

In other instances, a guest operating system may be specially designed or configured to execute on VMM 630 for efficiency. In these instances, the guest operating system is “aware” that it executes on a virtual machine monitor. In other words, VMM 630 may provide para-virtualization to a guest operating system in some instances.

A computer system process comprises an allotment of hardware processor time and an allotment of memory (physical and/or virtual), the allotment of memory being for storing instructions executed by the hardware processor, for storing data generated by the hardware processor executing the instructions, and/or for storing the hardware processor state (e.g., the content of registers) between allotments of the hardware processor time when the computer system process is not running. Computer system processes run under the control of an operating system and may run under the control of other programs being executed on the computer system.

Multiple threads may run within a process. Each thread also comprises an allotment of hardware processing time but shares access to the memory allotted to the process. The memory is used to store the content of processors between the allotments when the thread is not running. The term thread may also be used to refer to a computer system process in multiple threads that are not running.

### Hardware Overview

According to one embodiment of the invention, the techniques described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices or any other device that incorporates hard-wired and/or program logic to implement the techniques.

FIG. 7 is a block diagram that depicts an example computer system 700 upon which embodiments of the invention may be implemented. Computer system 700 includes a bus 702 or other communication mechanism for communicating information, and a processor 704 coupled with bus 702 for processing information. Computer system 700 also includes a main memory 706, such as a random access memory (RAM) or other dynamic storage device, coupled to bus 702 for storing information and instructions to be executed by processor 704. Main memory 706 also may be used for storing temporary variables or other intermediate information during execution of instructions to be executed by processor 704. Computer system 700 further includes a read only memory (ROM) 708 or other static storage device coupled to bus 702 for storing static information and instructions for processor 704. A storage device 710, such as a magnetic disk or optical disk, is provided and coupled to bus 702 for storing information and instructions.

Computer system 700 may be coupled via bus 702 to a display 712, such as a cathode ray tube (CRT), for displaying information to a computer user. Although bus 702 is illustrated as a single bus, bus 702 may comprise one or more buses. For example, bus 702 may include without limitation a control bus by which processor 704 controls other devices within computer system 700, an address bus by which processor 704 specifies memory locations of instructions for execution, or any other type of bus for transferring data or signals between components of computer system 700.

An input device 714, including alphanumeric and other keys, is coupled to bus 702 for communicating information and command selections to processor 704. Another type of user input device is cursor control 716, such as a mouse, a trackball, or cursor direction keys for communicating direction information and command selections to processor 704 and for controlling cursor movement on display 712. This input device typically has two degrees of freedom in two axes, a first axis (e.g., x) and a second axis (e.g., y), that allows the device to specify positions in a plane.

Computer system 700 may implement the techniques described herein using customized hard-wired logic, one or more ASICs or FPGAs, firmware and/or program logic or computer software which, in combination with the computer system, causes or programs computer system 700 to be a special-purpose machine. According to one embodiment of the invention, those techniques are performed by computer system 700 in response to processor 704 executing one or more sequences of one or more instructions contained in main memory 706. Such instructions may be read into main memory 706 from another computer-readable medium, such as storage device 710. Execution of the sequences of instructions contained in main memory 706 causes processor 704 to perform the process steps described herein. In alternative embodiments, hard-wired circuitry may be used in place of or in combination with software instructions to implement the invention. Thus, embodiments of the invention are not limited to any specific combination of hardware circuitry and software.

The term “computer-readable medium” as used herein refers to any medium that participates in providing data that causes a computer to operate in a specific manner. In an embodiment implemented using computer system 700, various computer-readable media are involved, for example, in providing instructions to processor 704 for execution. Such a medium may take many forms, including but not limited to, non-volatile media and volatile media. Non-volatile media includes, for example, optical or magnetic disks, such as storage device 710. Volatile media includes dynamic memory, such as main memory 706. Common forms of computer-readable media include, for example, a floppy disk, a flexible disk, hard disk, magnetic tape, or any other magnetic medium, a CD-ROM, any other optical medium, a RAM, a PROM, and EPROM, a FLASH-EPROM, any other memory chip or memory cartridge, or any other medium from which a computer can read.

Various forms of computer-readable media may be involved in carrying one or more sequences of one or more instructions to processor 704 for execution. For example, the instructions may initially be carried on a magnetic disk of a remote computer. The remote computer can load the instructions into its dynamic memory and send the instructions over a telephone line using a modem. A modem local to computer system 700 can receive the data on the telephone line and use an infra-red transmitter to convert the data to an infra-red signal. An infra-red detector can receive the data carried in the infra-red signal and appropriate circuitry can place the data on bus 702. Bus 702 carries the data to main memory 706, from which processor 704 retrieves and executes the instructions. The instructions received by main memory 706 may optionally be stored on storage device 710 either before or after execution by processor 704.

Computer system 700 also includes a communication interface 718 coupled to bus 702. Communication interface 718 provides a two-way data communication coupling to a network link 720 that is connected to a local network 722. For example, communication interface 718 may be an integrated services digital network (ISDN) card or a modem to provide a data communication connection to a corresponding type of telephone line. As another example, communication interface 718 may be a local area network (LAN) card to provide a data communication connection to a compatible LAN. Wireless links may also be implemented. In any such implementation, communication interface 718 sends and receives electrical, electromagnetic or optical signals that carry digital data streams representing various types of information.

Network link 720 typically provides data communication through one or more networks to other data devices. For example, network link 720 may provide a connection through local network 722 to a host computer 724 or to data equipment operated by an Internet Service Provider (ISP) 726. ISP 726 in turn provides data communication services through the world wide packet data communication network now commonly referred to as the “Internet” 728. Local network 722 and Internet 728 both use electrical, electromagnetic or optical signals that carry digital data streams.

Computer system 700 can send messages and receive data, including program code, through the network(s), network link 720 and communication interface 718. In the Internet example, a server 730 might transmit a requested code for an application program through Internet 728, ISP 726, local network 722 and communication interface 718. The received code may be executed by processor 704 as it is received, and/or stored in storage device 710, or other non-volatile storage for later execution.

### Cloud Computing

The term “cloud computing” is generally used herein to describe a computing model which enables on-demand access to a shared pool of computing resources, such as computer networks, servers, software applications, and services, and which allows for rapid provisioning and release of resources with minimal management effort or service provider interaction.

A cloud computing environment (sometimes referred to as a cloud environment or a cloud) can be implemented in a variety of different ways to best suit different requirements. For example, in a public cloud environment, the underlying computing infrastructure is owned by an organization that makes its cloud services available to other organizations or to the general public. In contrast, a private cloud environment is generally intended solely for use by or within a single organization. A community cloud is intended to be shared by several organizations within a community, while a hybrid cloud comprises two or more types of cloud (e.g., private, community, or public) that are bound together by data and application portability.

Generally, a cloud computing model enables some of those responsibilities which previously may have been provided by an organization's own information technology department to instead be delivered as service layers within a cloud environment for use by consumers (either within or external to the organization, according to the cloud's public/private nature). Depending on the particular implementation, the precise definition of components or features provided by or within each cloud service layer can vary, but common examples include Software as a Service (SaaS), in which consumers use software applications that are running upon a cloud infrastructure, while a SaaS provider manages or controls the underlying cloud infrastructure and applications. Platform as a Service (PaaS), in which consumers can use software programming languages and development tools supported by a PaaS provider to develop, deploy, and otherwise control their own applications, while the PaaS provider manages or controls other aspects of the cloud environment (i.e., everything below the run-time execution environment). Infrastructure as a Service (IaaS), in which consumers can deploy and run arbitrary software applications and/or provision processing, storage, networks, and other fundamental computing resources, while an IaaS provider manages or controls the underlying physical cloud infrastructure (i.e., everything below the operating system layer). Database as a Service (DBaaS), in which consumers use a database server or Database Management System that is running upon a cloud infrastructure, while a DbaaS provider manages or controls the underlying cloud infrastructure, applications, and servers, including one or more database servers.

### Extensions and Alternatives

In the foregoing specification, embodiments have been described with reference to numerous specific details that may vary from implementation to implementation. Thus, the sole and exclusive indicator of what is, and is intended by the applicants to be, the invention is the set of claims that issue from this application, in the specific form in which such claims issue, including any subsequent correction. Hence, no limitation, element, property, feature, advantage or attribute that is not expressly recited in a claim should limit the scope of such claim in any way. The specification and drawings are, accordingly, to be regarded in an illustrative rather than a restrictive sense.

What is claimed is:

1. A system comprising a memory storing instructions which, when executed by one or more processors, cause performance of:
receiving measurement data from one or more first networked devices for a patient being monitored;
receiving mental condition data of the patient;
detecting, based on analyzing the measurement data from the one or more first networked devices, a change in a physical/physiological condition of the patient;
determining, based on the mental condition data and the change in the physical/physiological condition of the patient, that the change in the physical/physiological condition of the patient warrants communicating, to a first recipient, a first notification of the change in the physical/physiological condition of the patient;
communicating, toward the first recipient, the first notification of the change in the physical/physiological condition of the patient.

2. The system of claim 1, wherein the mental condition data was generated based on applying natural language processing techniques to voice or text input provided by the patient.

3. The system of claim 1, wherein the mental condition data is obtained from electronic health records or electronic medical records of the patient.

4. The system of claim 1, wherein the memory further stores instructions which, when executed by the one or more processors, cause performance of:
prior to communicating, toward the first recipient, the notification of the change in the physical/physiological condition of the patient, identifying, based on the mental condition data and the change in the physical/physiological condition, one or more suspected causes for the change in the physical/physiological condition of the patient;
requesting context data that confirms or eliminates a suspected cause of the one or more suspected causes.

5. The system of claim 4, wherein requesting context data that confirms or eliminates a suspected cause of the one or more suspected causes comprises:
initiating establishment of a two-way communication between the patient and the first recipient, or
ordering a medical test that provides insight about the change in the physical/physiological condition of the patient.

6. The system of claim 4, wherein the memory further stores instructions which, when executed by the one or more processors, cause performance of:
receiving the context data from one or more second networked devices that are different from the one or more first networked devices.

7. The system of claim 1, wherein the memory further stores instructions which, when executed by the one or more processors, cause performance of:
determining that the mental condition data warrants communicating, to a second recipient, a second notification of a change in a mental condition of the patient;
communicating, toward the second recipient, the second notification of the change in the mental condition of the patient.

8. A computer-readable medium storing instructions which, when executed by one or more processors, cause performance of:
receiving measurement data from one or more first networked devices for a patient being monitored;
receiving mental condition data of the patient;
detecting, based on analyzing the measurement data from the one or more first networked devices, a change in a physical/physiological condition of the patient;
determining, based on the mental condition data and the change in the physical/physiological condition of the patient, that the change in the physical/physiological condition of the patient warrants communicating, to a first recipient, a first notification of the change in the physical/physiological condition of the patient;
communicating, toward the first recipient, the first notification of the change in the physical/physiological condition of the patient.

9. The computer-readable medium of claim 8, wherein the mental condition data was generated based on applying natural language processing techniques to voice or text input provided by the patient.

10. The computer-readable medium of claim 8, wherein the mental condition data is obtained from electronic health records or electronic medical records of the patient.

11. The computer-readable medium of claim 8, further storing instructions which, when executed by the one or more processors, cause performance of:
prior to communicating, toward the first recipient, the notification of the change in the physical/physiological condition of the patient, identifying, based on the mental condition data and the change in the physical/physiological condition, one or more suspected causes for the change in the physical/physiological condition of the patient;
requesting context data that confirms or eliminates a suspected cause of the one or more suspected causes.

12. The computer-readable medium of claim 11, wherein requesting context data that confirms or eliminates a suspected cause of the one or more suspected causes comprises:
initiating establishment of a two-way communication between the patient and the first recipient, or
ordering a medical test that provides insight about the change in the physical/physiological condition of the patient.

13. The computer-readable medium of claim 11, further storing instructions which, when executed by the one or more processors, cause performance of:
receiving the context data from one or more second networked devices that are different from the one or more first networked devices.

14. The computer-readable medium of claim 8, further storing instructions which, when executed by the one or more processors, cause performance of:
determining that the mental condition data warrants communicating, to a second recipient, a second notification of a change in a mental condition of the patient;
communicating, toward the second recipient, the second notification of the change in the mental condition of the patient.

15. A computer-implemented method comprising:
receiving measurement data from one or more first networked devices for a patient being monitored;
receiving mental condition data of the patient;
detecting, based on analyzing the measurement data from the one or more first networked devices, a change in a physical/physiological condition of the patient;
determining, based on the mental condition data and the change in the physical/physiological condition of the patient, that the change in the physical/physiological condition of the patient warrants communicating, to a first recipient, a first notification of the change in the physical/physiological condition of the patient;
communicating, toward the first recipient, the first notification of the change in the physical/physiological condition of the patient.

16. The method of claim 15, wherein the mental condition data was generated based on applying natural language processing techniques to voice or text input provided by the patient.

17. The method of claim 15, wherein the mental condition data is obtained from electronic health records or electronic medical records of the patient.

18. The method of claim 15, further comprising:
prior to communicating, toward the first recipient, the notification of the change in the physical/physiological condition of the patient, identifying, based on the mental condition data and the change in the physical/physiological condition, one or more suspected causes for the change in the physical/physiological condition of the patient;
requesting context data that confirms or eliminates a suspected cause of the one or more suspected causes.

19. The method of claim 18, further comprising:
receiving the context data from one or more second networked devices that are different from the one or more first networked devices.

20. The method of claim 15, further comprising:
determining that the mental condition data warrants communicating, to a second recipient, a second notification of a change in a mental condition of the patient;
communicating, toward the second recipient, the second notification of the change in the mental condition of the patient.
